(19) United States
(12) Patent Application Publication
(10) Pub. No.: US 2023/0285409 A1
(43) Pub. Date: Sep. 14, 2023

(54) METHODS OF USING RHO KINASE INHIBITORS TO TREAT VASCULAR DEMENTIA

(71) Applicant: Woolsey Pharmaceuticals, Inc., St. Petersburg, FL (US)

(72) Inventors: Thomas MACALLISTER, Arlington, VA (US); Sven JACOBSON, New York, NY (US)

(21) Appl. No.: 18/001,514

(22) PCT Filed: Jan. 8, 2021
(86) PCT No.: PCT/US2021/012590

§ 371 Date: Dec. 12, 2022

Related U.S. Application Data
(60) Provisional application No. 63/046,173, filed on Jun. 30, 2020.
(60) Provisional application No. 63/039,141, filed on Jun. 15, 2020.

(51) Int. Cl. A61K 31/551; A61K 9/00; A61P 25/28

(52) U.S. Cl. CPC A61K 31/551; A61K 9/0053; A61P 25/28

(57) ABSTRACT

Disclosed are methods of treating patients with VaD using a rho kinase inhibitor. A preferred rho kinase inhibitor used according to the invention is fasudil, which is typically administered orally in a total daily dose of 70 - 180 mg. A preferred dosing regimen involves administering the daily dose in three equal portions throughout the day. Preferred methods continue for more than one month and typically at least 2 or 3 or even 6 months or more. Some preferred methods do not treat mild cognitive impairment and patients have and MMSE score of ≤ 23.

### CROSS-REFERENCE TO RELATED APPLICATIONS

This application claims priority to U.S. Provisional application no. 63/039,141, filed on Jun. 15, 2020, and to U.S. Provisional application no. 63/046,173, filed on Jun. 30, 2020, the disclosures of which are incorporated herein in their entireties.

### BACKGROUND OF THE INVENTION

Among the dementias, vascular dementia (VaD) is differentiated from other forms of dementia by the presence of one or more vascular causes in the general absence of other pathologies. Specifically, VaD is not a neurodegenerative disease, unlike all other types of dementia (Salardini 2019). Uniquely, the pathophysiology of VaD is not linked to an underlying proteinopathy.

The two main subtypes vascular dementia are i) large cortical infarction or multi-infarct dementia (MID) and ii) small vessel disease-related dementia or subcortical vascular dementia. Two patients treated with a Rho Kinase (ROCK) inhibitor, fasudil, by Kamei 1996 both had subcortical vascular dementia, which is caused by disruption of the vasculature in the subcortical white matter-rich areas of the brain and one patient had a hemorrhage. The International Classification of Diseases (10th revision) (ICD-10) criteria for vascular dementia explicitly identifies subcortical vascular dementia as a subgroup (Wetterling et al. 1994). Subcortical vascular dementia incorporates the old entities “lacunar state” and “Binswanger disease” and relates to small vessel disease and hypoperfusion resulting in focal and diffuse ischemic white matter lesion and incomplete ischemic injury (Erkinjuntti, 1997). On the other hand, most vascular dementia patients suffer from the first type [large cortical infarction or multi-infarct dementia (MID)], affecting the cortical regions of the brain, and present with different defects that result from very different pathophysiological processes.

The etiologies, pathologies and symptoms of sub-cortical and cortical vascular dementias are well characterized. Large vessel cortical strokes and subcortical small vessel disease tend to produce different kinds of deficits. Characteristic symptoms of subcortical dementia typically include forgetfulness, slowing of thought processes, mild intellectual impairment, apathy, inertia, depression (sometimes with irritability), loss of recall ability, and the inability to manipulate knowledge. Additionally, subcortical dementia patients have mood disorders. Other behavioral abnormalities like repetitive and compulsive behavior occur in some patients suffering from subcortical dementia. Generally, sub-cortical dementia presentation is more subtle and temporally progressive, often described as defects in executive function in sub-cortical dementia. This includes deficits in speed and “strategic” processing (i.e., attention, planning, and monitoring) in tasks such as memory tasks. In contrast, cortical vascular dementia is associated with aphasia, apraxia and amnesia.

Memory is impaired in both sub-cortical and cortical vascular dementia. But in cortical vascular dementias, the recall abnormality is due to a failure to encode information properly or decay of memory consolidation. Behavioral changes may include apathy, lack of spontaneity, and perseveration. In contrast, in subcortical disorders exhibit deficits in spontaneous recall, but encoding and storage are largely preserved, and recollection can be aided. Subcortical dementia is characterized by a relatively mild retrograde amnesia that equally affects all time periods because here there is faulty retrieval of successfully stored information. It is the recall deficit that results in wayfinding problems in sub-cortical vascular dementia.

Sub-cortical and cortical dementia are differentially diagnosed. White matter hyperintensities (i.e., sub-cortical) are considered to result from cerebral small vessel disease, especially at larger volumes. This damage can be quantified using the Fazekas scale: 0 (no lesions); 1 (punctiform lesions); 2 (early confluent lesions); and 3 (confluent lesions). A Fazekas score of 1 can be considered normal, whereas scores 2 and 3 indicate the presence of small vessel disease. A score of 3 is abnormal at any age. The presence of confluent lesions in the frontal and parietal lobes is indicative of a large white matter pathology (>25%) and can be used in making a diagnosis of (subcortical) vascular dementia. Lacunar infarcts involving multiple basal ganglia and the frontal white matter, as well as bilateral thalamic lesions are also diagnostic of subcortical vascular dementia.

Strategic large vessel infarctions can indicate cortical dementia when they involve the following territories: bilateral anterior cerebral artery, paramedian thalamic, inferior medial temporal lobe, parieto-temporal and temporo-occipital association areas and angular gyrus, superior frontal and parietal watershed areas in the dominant hemisphere.

A central issue with interventions that target dementia is that of association versus causation. In order for an intervention to work in treating a disease, it must interrupt the chain of causation. AD, the most common form of dementia, provides a very instructive case. The two characteristic pathological findings of AD are the extracellular amyloid plaques and inter-neuronal neurofibrillary tangles (NFT).

While Aβ, tau and neuroinflammation are certainly associated with AD, is it not clear they are involved in causation and thus, it is unclear that affecting any of these will have any therapeutic benefit in treating the disease. Based on understanding the familial disease, it is believed that Aβ starts the process of neurodegeneration by inducing Tau pathology, neuroinflammation and finally the neuronal loss that leads to cognitive decline. In other words, Aβ is at the beginning of the causality chain. Stopping Aβ pathology should stop the disease and, so far, most therapeutic approaches have targeted Aβ.

Despite the overwhelming literature showing the promise of targeting Aβ in animal models, however, there have been no products that have been shown to work in AD (Ceyzériat et al., 2020). These failures include, notably among many, Anti-Aβ42 + Freud’s adjuvant, Bapineuzumab, Solanezumab, Aducanumab, Verubecestat, Lanabecestat, Atabecestat, CNP520, Elenbecestat, γ-Secretase inhibitors, Bryostatin and PBT2.

Tau is a less likely target because of the evidence that it is downstream of Aβ, and thus is not causative, and so trials have been less frequent. Notably, of 15 trial targeting tau that have been initiated, already four of them have been stopped for futility.

The role of neuroinflammation, the third putative interventional target, in AD is unclear, likely being beneficial in early-stage disease, but possibly evolving adversely by participating in a loop of pro-inflammatory cytokine production and oxidative stress. While epidemiological studies have suggested that treatment with nonsteroidal anti-inflammatory drugs (NSAIDs) reduce the risk of developing AD and they can decrease amyloid load in transgenic models, to date prospective studies testing anti-inflammatory drugs have shown no beneficial effect on cognition in AD. Studies targeting neuroinflammation are ongoing, but early results are not promising. Neflamapimod, a selective inhibitor of p38 mitogen-activated protein kinase showed efficacy in an animal model, but it had no effect on Aβ deposition in humans and failed its primary endpoint of improving episodic memory in Phase 2, despite reducing tau in the cerebrospinal fluid.

In view of the number of clinical failures of compounds that seemed promising in animal models, a grave degree of skepticism should be applied in interpreting animal data. Even aside from the obvious issues of differences in brain complexity between rodents and humans, many of the existing models bear only a passing resemblance to the human condition. Many things can cause neural degeneration in animals and many putative drugs can halt that neural degeneration, but the underlying pathophysiology and chain of causation is unknown and it is there that a disease modifying intervention must act. It is crucial, therefore, that animal models, with their known deficiencies in the best of cases, as closely resemble the human disease as possible, in both pathology and clinical presentation.

There are a number of publications looking at the use of rho kinase inhibitors in various models of AD/dementia. Most models are deficient in basic properties and none of these purport to be models of vascular dementia. Some models involve the direct induction of neurotoxicity with agents like streptozotocin or even by direct injection of amyloid-beta into the brain. While these models may exhibit certain AD-like properties, they are just models of neural degeneration and cannot predict treatment of AD itself. Even the transgenic models are deficient. For example, there are a number of transgenic mice that only develop amyloid plaques without NFTs, such as the APP/PS-1 mouse, perhaps the most widely reported transgenic model. There are also mice that develop tauopathies, without amyloid plaques, such as the rTG4510 tau mouse. AD is characterized by the presence of both. Some publications use unrealistic routes of administration (e.g., intraventricular injection) and many do not use appropriate dosing. In this regard, standard formulas exist for converting doses used in animals to the same dose in humans. Human equivalent dose can be calculated, for example, using Table 1 of Nair & Jacob2016), which are the same conversions used by the US FDA. Becker 2008) discusses the criticality of dose in successful AD drug development and points to it as a failure point in AD drug development.

Published literature exists in which fasudil is administered in animal models of dementia. But these studies are deficient for many of the same reasons. Namely, the animal models do not faithfully recapitulate human disease, partly due to species differences in neuroanatomy (Sasaguri 2017) and partly due to the deficient basic pathological bases of the models, described above. In addition, some fail to use physiologically relevant doses and, importantly, no outcomes relevant to wandering were measured in any of them. It is important also to note that the hallmark of onset in the paradigmatic cortical dementia, AD, is the failure of semantic memory, which cannot be measured in any animal model and so all animal models share this deficiency as well. For example, Hamano et al., 2019, administered 12 mg/kg/day (68 mg HED) to rTG4510 tau transgenic mice and measured only tau phosphorylation/cleavage and oligomers, but no outcomes. Elliott 2018 used a triple transgenic mouse model (APP Swedish, MAPT P301L, and PSEN1 M146V) and observed reduce ß-amyloid plaques in vivo at a dose of 10 mg/kg/day (intraperitoneally) fasudil (57 mg HED). Sellers 2018 used the AB42 mouse model and administered fasudil intraperitoneally at a dose of 10 mg/kg BID (226 mg HED) but monitored only ß-amyloid dendritic spine loss. Couch et al. 2010 used intraventricular infusion and observed effects on dendritic branching and no outcomes relevant to wandering. Putting aside the absence of any behavioral outcomes in these references, intraventricular administration is not a therapeutic option for humans. Yu 2017 and Hou 2012 administered fasudil at 5 and 10 mg/kg/day intraperitoneally to APP/PS1 transgenic mice (70, 140 mg HED) and streptozotocin rats (226 mg HED), respectively and observed that latency distance and quadrant time were improved in the Morris water maze (a model for spatial learning and memory, not wandering). It should be noted that there are streptozotocin models of VaD, but those involve the induction of diabetes and the vascular disturbances that result from that condition. The model of Yu 2017 and Hou 2012 is a neurotoxicity model (involving injecting streptozotocin into the brain) and completely unrelated to models of VaD.

Conflicting reports to the above also exist. For example, Turk 2018 (dissertation) used triple transgenic mice and did not observe improvements in spatial memory at 10 or 12 months of age with fasudil administered in water at 30 mg/kg and 100 mg/kg.

Based on currently available animal modeling, different therapeutic strategies targeting the pathological hallmarks of dementia have been tested but have failed to show any beneficial effects in humans. At present, available medications are limited to acetylcholinesterase inhibitors and N-methyl-D-aspartate (NMDA) receptor antagonists, which show only modest improvements in some cognitive symptoms. No existing or even proposed therapies address the problem of wandering in dementia, which is not treated by the foregoing approved therapeutics. There exists a significant unmet need to provide new therapies that show benefit in humans, not just animals.

Kamei (1996a) reported on using fasudil in two patients with wandering due to VaD. The patients were treated by the investigator for wandering following participating in a chronic stroke study where they were treated with fasudil. One patient was diagnosed with Binswanger-type cerebral infarction, confirmed by MRI imaging. Prior to treatment, the patient had a history of more than 3.5 years of wandering symptoms, consisting primarily of wayfinding problems. The patient could not find his way home. Then, for about a year-and-a-half prior to beginning treatment, the patient was regularly eloping approximately 2-3 times per week. Within weeks of beginning treatment, wandering symptoms disappeared and remained absent for the duration of treatment. When the patient was removed from treatment, wandering symptoms reappeared within weeks. Upon re-treatment, wandering again resolved. The other patient was diagnosed with sequelae of cerebral bleeding and multiple lacunar infarctions, confirmed by MRI, and a diagnosis of “lacunar dementia” (a synonym of Binswanger’s; Román 1985) Approximately 5 months after the hemorrhage, the patient began exhibiting wayfinding symptoms, beginning with several episodes of losing his way with frequency increasing to 2-3 times per week over several months. Wayfinding symptoms disappeared quickly and remained absent for the duration of treatment, returning each time treatment was stopped.

Moreover, both Kamei 1996a patients were sporadic wanderers, wandering 2-3 days per week and they displayed primarily a wayfinding defect (getting lost), and no other problematic behavior. Kamei also published another paper in 1996 (Kamei 1996b) with substantially the same findings. Prior to these publications, Kamei filed a patent application in Japan (Patent Application 6-293643) based on the same two patients in the publication and a third patient. It should also be noted that Kamei 1996a presented two cognitive measures, the Mini Mental State Exam (MMSE; Folstein 1975) and the Hasegawa Dementia Score (HDS), which are very similar and usually yield very similar results. In fact, the HDS usually scores dementia patients as more severe than the MMSE (Kim 2005), yet not only were the MMSE scores in Kamei 1996a consistently worse than the HDS, the different scores lead to a dramatically different understanding of the patient population. The HDS suggests that the patients had only mild dementia, whereas the MMSE suggest that they are moderately to severely demented (wandering is known to be associate with more advanced/severe dementia).

There is no evidence that the work of Kamei in subcortical vascular dementia can be extrapolated to cortical forms of dementia or to non-vascular forms of subcortical dementia, nor that it can be extrapolated to persistent wanderers or wanderers without a wayfinding defect.

Zhang 2012 reports on a study of 90 VaD patients, half treated with intravenous fasudil and the other half treated with intravenous ligustrazine. The trial was randomized, but not blinded or placebo-controlled. Patients were included in accordance with the VaD diagnosis criteria of the American Academy of Neurology and NINDS-AIREN. Maximum mini mental state examination (MMSE) scores were required and scaled with educational level: illiterate <17 points; primary school education <20 points; high school education or higher <24 points. A Hachinski Ischemic Scale >7 points was required. Patients diagnosed with other forms of dementia and patients with consciousness disturbances or other mental disorders (e.g. depression) were also excluded. Fasudil treated patients received 30 mg of drug by intravenous infusion over 30 minutes every day for 2 weeks, when drug was stopped for 2 days and then resumed for an additional 2 weeks. The investigators reported a 9-point (mean or median, it is unclear) improvement in MMSE over baseline and a 7-point differential versus control.

There are a number of reasons to question the reliability of the Zhang results. First off, the non-blinded trial design means it was no secret which was the test group and which was the control.

It is well known that subjective cognitive assessments can be affected by the rater and without blinded raters there will be a tendency to rate in accordance with the expectation a therapy will work. Moreover, there is a more significant reason to discount these data. The authors use an activities of daily living scale (ADL) in order to assess function. The authors disclosed that a score of > 26 is considered functionally impaired. The treated subjects were clearly profoundly impaired with a (mean or median, it is unclear) ADL score of 54. It is important to note that dementia is not mere cognitive decline; rather, it is cognitive decline severe enough to meaningfully impact function (ie, ADL). Thus, with a mean MMSE of about 17 (considered moderate to severely cognitively impaired) and an ADL score of 54 (very functionally impaired), the Zhang cohort is clearly demented. On the other hand, after treatment, the MMSE score improves to nearly 27, meaning they no longer cognitively impaired - all in the span of a month of discontinuous treatment. On face, this is a truly remarkable result. However, the ADL score reduces only to 36, meaning the patients remained very functionally impaired based on a threshold of 26. Because the cognitive impairment results in the functional impairment in dementia, a dramatic increase in cognition in the absence of a dramatic increase in function suggests that the cognitive improvement is a result of a biased assessment, which is encouraged by the poor study design.

The evidence in Kamei also gives rise to some skepticism. First of all, it is based on case studies, which are neither controlled, nor blinded and so there is the same risk of assessment bias as in Zhang. That said, the cognitive improvements were much more modest (about 3 points rather than 9) and the patients did not jump from a cognitive status suggesting they started severely demented and then the dementia disappeared with treatment; rather, the MMSE scores suggest they started severely demented and remained severely demented after treatment, with only mild improvements in certain types of memory. The functional improvement was limited to the disappearance of wandering, which principally manifested in getting lost; however, it is interesting to note that Kamei reported no improvement in spatial orientation. If patients were getting lost and that stopped on drug treatment, one would expect that orientation as to space would improve, but it clearly did not. It is also notable that Kamei used two different doses (30 mg and 60 mg per day) and there was no dose response, meaning the low and high dose appeared to work to the same extent.

The present invention is based in part on the discovery of an optimum dosing regimen for fasudil when used in treating patients with VaD, which is higher than the doses of either Zhang or Kamei, but is limited at the upper end by renal disturbances not reported in the prior art.

### SUMMARY OF THE INVENTION

The invention relates to the treatment of vascular dementia with a rho kinase inhibitor. According to the invention, a preferred rho kinase inhibitor is fasudil, which is preferably administered orally in an amount of 70-140 mg daily.

In certain embodiments, patients may have subcortical or cortical vascular dementia. In other embodiments, patients may have mixed dementia, with vascular dementia in addition to pathologies and/or symptoms associated with other forms of dementia. Preferred methods treat more a minimum of more than 1 months and generally for a minimum of 4 or even 6 months.

Certain embodiments contemplate a minimum dose of 70 mg per day with the upper limit of dosing is determined by monitoring kidney function.

In one embodiment, the patient to be treated suffers from cerebral autosomal dominant arteriopathy with subcortical infarcts and leukoencephalopathy (CADISIL). CADISIL is a genetic, heritable disorder caused by autosomal dominant mutations in the Notch3 gene. These mutations result in accumulation of an abnormal Notch 3 protein at the cytoplasmic membrane of vascular smooth muscle cells in cerebral and extracerebral vessels. The abnormal Notch 3 impairs the survival of vascular smooth muscle cells surrounding the blood vessels, causing them to gradually die, resulting in arteriopathy. MRIs show white matter lesions of various sizes, concentrated around the basal ganglia, peri-ventricular white matter, and pons.

CADISIL patients suffer from ischemic strokes, migraine headaches, and transient ischemic attacks usually beginning in their mid- to late thirties or early forties. The disease progresses to subcortical VaD, as the sub-cortical strokes result in progressive loss of brain function and cognitive decline, usually by age 65.

In a specific embodiment, the CADASIL patient to be treated is asymptomatic but contains a Notch3 mutation, is diagnosed by a skin biopsy to detect changes in small arteries or is diagnosed by MRI.

In another embodiment, treatment with fasudil delays the progression to dementia in a CADASIL patient.

Some embodiments are understood to exclude certain patients, for example, patients with evidence of a hemorrhagic lesion, patients with pseudobulbar affect and/or patients with hypertension. In other embodiments, however, such patients are not excluded.

Contemplated methods seek to improve cognition, which may include improving executive function, and/or activities of daily living in a patient with vascular dementia.

While the treatment of patients with mild cognitive impairment or vascular cognitive impairment that is not severe enough to be considered dementia, most preferred methods contemplate treatings patients with an MMSE score of ≤23.

### DETAILED DESCRIPTION OF THE INVENTION

### ROCK Inhibitors

The inventive methods contemplate the administration of a rho kinase (ROCK) inhibitor in the treatment of a disease or condition. Two mammalian ROCK homologs are known, ROCK1 (aka ROKβ, Rho-kinase β, or p160ROCK) and ROCK2 (aka ROKα) (Nakagawa 1996). In humans, the genes for both ROCK1 and ROCK2 are located on chromosome 18. The two ROCK isoforms share 64% identity in their primary amino acid sequence, whereas the homology in the kinase domain is even higher (92%) (Jacobs 2006; Yamaguchi 2006). Both ROCK isoforms are serine/threonine kinases and have a similar structure.

A large number of pharmacological ROCK inhibitors are known (Feng, LoGrasso, Defert, & Li, 2015). Isoquinoline derivatives are a preferred class of ROCK inhibitors. The isoquinoline derivative fasudil was the first small molecule ROCK inhibitor developed by Asahi Chemical Industry (Tokyo, Japan). The characteristic chemical structure of fasudil consists of an isoquinoline ring, connected via a sulphonyl group to a homopiperazine ring. Fasudil is a potent inhibitor of both ROCK isoforms. In vivo, fasudil is subjected to hepatic metabolism to its active metabolite hydroxyfasudil (aka, M3). Other examples of isoquinolone derived ROCK inhibitors include dimethylfasudil and ripasudil.

Other preferred ROCK inhibitors are based on based on 4-aminopyridine structures. These were first developed by Yoshitomi Pharmaceutical (Uehata et al., 1997) and are exemplified by Y-27632. Still other preferred ROCK inhibitors incude indazole, pyrimidine, pyrrolopyridine, pyrazole, benzimidazole, benzothiazole, benzathiophene, benzamide, aminofurazane, quinazoline, and boron derivatives (Feng et al., 2015). Some exemplary ROCK inhibitors are shown below:

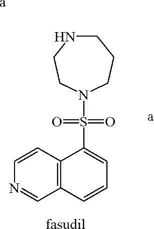

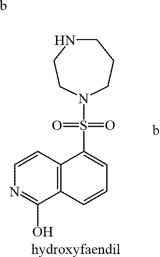

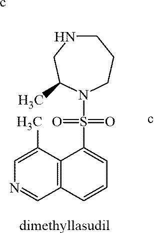

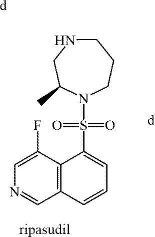

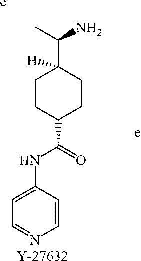

ROCK inhibitors according to the invention may have more selective activity for either ROCK1 or ROCK2 and will usually have varying levels of activity on PKA, PKG, PKC, and MLCK. Some ROCK inhibitors may be highly specific for ROCK1 and/or ROCK2 and have much lower activity against PKA, PKG, PKC, and MLCK.

A particularly preferred ROCK inhibitor is fasudil. Fasudil may be exist as a free base or salt and may be in the form of a hydrate, such as a hemihydrate. As used herein, unless specifically noted, the name of any active moiety, such as fasudil, should be considered to include all forms of the active moiety, including the free acid or base, salts, hydrates, polymorphs and prodrugs of the active moiety.

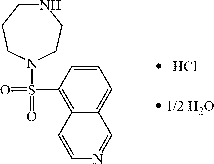

### Hexahydro-1-(5-Isoquinolinesulfonyl)-1H-1,4-Diazepine Monohydrochloride Hemihydrate

Fasudil is a selective inhibitor of protein kinases, such as ROCK, PKC and MLCK and treatment results in a potent relaxation of vascular smooth muscle, resulting in enhanced blood flow (Shibuya 2001). A particularly important mediator of vasospasm, ROCK induces vasoconstriction by phosphorylating the myosin-binding subunit of myosin light chain (MLC) phosphatase, thus decreasing MLC phosphatase activity and enhancing vascular smooth muscle contraction. Moreover, there is evidence that fasudil increases endothelial nitric oxide synthase (eNOS) expression by stabilizing eNOS mRNA, which contributes to an increase in the level of the potent vasodilator nitric oxide (NO), thereby enhancing vasodilation (Chen 2013).

Fasudil has a short half-life of about 25 minutes, but it is substantially converted in vivo to its 1-hydroxy (M3) metabolite. M3 has similar effects to its fasudil parent molecule, with slightly enhanced activity and a half-life of about 8 hours (Shibuya 2001). Thus, M3 is likely responsible for the bulk of the in vivo pharmacological activity of the molecule. M3 exists as two tautomers, depicted below:

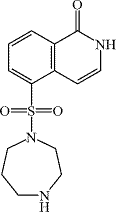 [SMILES: O=c1[nH]ccc2c(S(=O)(=O)N3CCCNCC3)cccc12]

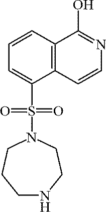 [SMILES: O=S(=O)(c1cccc2c(O)nccc12)N1CCCNCC1]

The ROCK inhibitors used in the invention, such as fasudil, include pharmaceutically acceptable salts and hydrates. Salts that may be formed via reaction with inorganic and organic acid. Those inorganic and organic acids are included as following: hydrochloric acid, hydrobromide acid, hydriodic acid, sulphuric acid, nitric acid, phosphoric acid, acetic acid, maleic acid, maleic acid, maleic acid, oxalic acid, oxalic acid, tartaric acid, malic acid, mandelic acid, trifluoroacetic acid, pantothenic acid, methane sulfonic acid, or para-toluenesulfonic acid.

### Pharmaceutical Compositions

Pharmaceutical compositions of ROCK inhibitors usable in the are generally oral and may be in the form of tablets or capsules and may be immediate-release formulations (ie, those in which no elements of the formulation are designed to substantially control or retard the release of the ROCK inhibitor upon administration) or may be controlled- or extended-release formulations, which may contain pharmaceutically acceptable excipients, such as corn starch, mannitol, povidone, magnesium stearate, talc, cellulose, methylcellulose, carboxymethylcellulose and similar substances. A pharmaceutical composition comprising a ROCK inhibitor and/or a salt thereof may comprise one or more pharmaceutically acceptable excipients, which are known in the art. Formulations include oral films, orally disintegrating tablets, effervescent tablets and granules or beads that can be sprinkled on food or mixed with liquid as a slurry or poured directly into the mouth to be washed down.

Pharmaceutical compositions containing ROCK inhibitors, salts and hydrates thereof can be prepared by any method known in the art of pharmaceutics. In general, such preparatory methods include the steps of bringing a ROCK inhibitor or a pharmaceutically acceptable salt thereof into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. The composition used in accordance with the methods of the present invention may comprise between 0.001% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

In certain embodiments, the pharmaceutical composition used in the methods of the present invention may comprise a diluent. Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

In certain embodiments, the pharmaceutical composition used in the methods of the present invention may comprise a granulating and/or dispersing agent. Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM), sodium lauryl sulfate, quaternary ammonium compounds, and mixtures thereof.

In certain embodiments, the pharmaceutical composition used in the methods of the present invention may comprise a binding agent. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM.RTM.), and larch arabogalactan), alginates, polyethylene oxide, polyethylene glycol, inorganic calcium salts, silicic acid, polymethacrylates, waxes, water, alcohol, and/or mixtures thereof.

In certain embodiments, the pharmaceutical composition used in the methods of the present invention may comprise a preservative. Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

In certain embodiments, the pharmaceutical composition used in the methods of the present invention may comprise an antioxidant. Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

In certain embodiments, the pharmaceutical composition used in the methods of the present invention may comprise a chelating agent. Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

In certain embodiments, the pharmaceutical composition may comprise a buffering agent together with the ROCK inhibitor or the salt thereof. Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer’s solution, ethyl alcohol, and mixtures thereof.

In certain embodiments, the pharmaceutical composition used in the methods of the present invention may comprise a lubricating agent. Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

In other embodiments, the pharmaceutical composition of containing a ROCK inhibitor or salt thereof will be administered as a liquid dosage form. Liquid dosage forms for oral and parenteral administration include pharmaceutically acceptable emulsions, microemulsions, solutions, suspensions, syrups, and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor™, alcohols, oils, modified oils, glycols, polysorbates, cyclodextrins, polymers, and mixtures thereof.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Some compositions of the invention relate to extended- or controlled-release formulations. These may be, for example, diffusion-controlled products, dissolution-controlled products, erosion products, osmotic pump systems or ionic resin systems. Diffusion-controlled products comprise a water-insoluble polymer which controls the flow of water and the subsequent egress of dissolved drug from the dosage from. Dissolution-controlled products control the rate of dissolution of the drug by using a polymer that slowly solubilizes or by microencapsulation of the drug - using varying thicknesses to control release. Erosion products control release of drug by the erosion rate of a carrier matrix. Osmotic pump systems release a drug based on the constant inflow of water across a semi permeable membrane into a reservoir which contains an osmotic agent. Ion exchange resins can be used to bind drugs such that, when ingested, the release of drug is determined by the ionic environment within the gastrointestinal tract.

### Treatable Patients

The invention contemplates using rho kinase inhibitors in the treatment of patients with VaD. The contemplated therapy is believed to be disease-modifying and so the inventive methods specifically contemplate treating or alleviating the various clinical presentations and symptoms of the disease, along with improvements in markers of VaD. It is further contemplated that rho kinase inhibitors according to the invention can be used to treat mixed dementia, having pathologies not exclusively attributable to VaD and overlapping with other dementias, such as Alzheimer’s dementia (AD). All types/subtypes of VaD are treatable according to the invention, including VaD stemming from the various etiologies and pathologies described in the literature. The inventive methods are capable of reducing or eliminating the various symptoms and mood disorders associated with VaD.

The two main subtypes vascular dementia are i) large cortical infarction or multi-infarct dementia (MID) and ii) small vessel disease-related dementia or subcortical vascular dementia. Subcortical vascular dementia is caused by disruption of the vasculature in the subcortical white matter-rich areas of the brain. The International Classification of Diseases (10th revision) (ICD-10) criteria for vascular explicitly identifies subcortical vascular dementia as a subgroup (Wetterling et al. 1994). Subcortical vascular dementia therefore, incorporates the old entities “lacunar state” and “Binswanger disease” and relates to small vessel disease and hypoperfusion resulting in focal and diffuse ischemic white matter lesion and incomplete ischemic injury. (Erkinjuntti, 1997). On the other hand, most dementia patients (mostly non-VaD patients) suffer from the first type, affecting the cortical regions of the brain, and present with different defects that result from very different pathophysiological processes.

The etiologies, pathologies and symptoms of sub-cortical and cortical vascular dementias are well characterized. Large vessel cortical strokes and subcortical small vessel disease tend to produce different kinds of deficits. Characteristic symptoms of subcortical dementia typically include forgetfulness, slowing of thought processes, mild intellectual impairment, apathy, inertia, depression (sometimes with irritability), loss of recall ability, and the inability to manipulate knowledge. Additionally, subcortical dementia patients have mood disorders. Other behavioral abnormalities like repetitive and compulsive behavior occur in some patients suffering from subcortical dementia and can be improved with the inventive methods. Generally, sub-cortical dementia presentation is more subtle and temporally progressive, often described as defects in executive function in sub-cortical dementia, which may also improve with the inhibition of rho kinases according to the invention. This includes deficits in speed and “strategic” processing (i.e., attention, planning, and monitoring) in tasks such as memory tasks. In contrast, cortical vascular dementia is associated with aphasia, apraxia and amnesia, which also are improved by the methods of the invention.

The American Psychiatric Association differentiates between mild and major neurocognitive impairment:
Mild neurocognitive impairment is defined as a cognitive decline one to two standard deviations from normal on formal cognitive testing that does not interfere with independence and is not due to delirium or other medical or psychiatric disorder.
Major neurocognitive impairment is defined as a cognitive decline two standard deviations or more from normal on formal cognitive testing that does interferes with independence and is not due to delirium or other medical or psychiatric disorder.

While patients with any neurocognitive deficit may be treated according to the invention, VaD patients typically will have a major neurocognitive impairment according to these criteria, such that the impairment interferes with their independence. Impairment of independence can be assessed using a scale that measures activities of daily living (ADL), including scales like the Barthel Index and the Alzheimer’s Disease Cooperative Study ADL inventory. Often, patients treatable according to the invention will have restricted independence in that they are residents in an assisted living or a memory care facility and are not community- or home-dwelling due to their condition.

Diagnostic and Statistical Manual of Mental Disorders Fifth Edition (DSM-V) provides a useful framework for the identification of patients treatable according to the invention. The DSM-V provides definitions of dementia syndrome.

Dementia syndrome requires objective cognitive or behavioral impairment in at least two of the following: memory; reasoning and handling complex tasks; visuospatial abilities; language functions; and personality, behavior, or comportment. It also requires a decline from previous level of functioning and a functional impairment.

While some patients may have mixed pathology, true VaD is dementia is precipitated by a cardiovascular event, such as an ischemic or hemorrhagic stroke, or a chronic cardiovascular condition, such as Binswanger’s disease or lucunar dementia. True VaD patients can be readily identified using the criteria of the National Institute of Neurological Disorders and Stroke (NINDS) and the Association Internationale pour la Recherche et 1′ Enseignement en Neurosciences (AIREN) (the NINDS-AIREN criteria) (Wetterling 1996; Román 1993). The NINDS-AIREN criteria specifically require confirmation of vascular pathology using imaging. Thus, patients identified according to the NINDS-AIREN criteria are specifically included.

Another useful tool in identifying VaD patients is the Hachinski Ischemia Score, in which diagnosed stroke, rapid onset, fluctuating course, and focal signs and symptoms, all indicative of stroke, are more heavily weighted. According to Hachinski, the following features of patients with dementia are scored with two points: abrupt onset; fluctuating course; history of strokes; focal neurological symptoms; focal neurological signs. The following elements that are less likely to be related to a cardiovascular event (and thus VaD) are scored with one point each: emotional incontinence; stepwise deterioration; history of hypertension; nocturnal confusion; evidence of associated atherosclerosis; relative preservation of personality; depression; and somatic complaints. Typically, a score >7 would indicate the patient has VaD. Hence, patients treated in accordance with the invention will typically have an Hachinski score of > 7 and patients with a Hachinski score of ≤ 7 would be excluded.

In one aspect, the invention excludes patients with pure AD, which can be identified by routine imaging as having a lack of vascular pathology and/or a lack of cardiovascular risk factors and cardiovascular events in their medical history. On the other hand, patients with mixed dementia, including any with suspect lesions on imaging, a known cardiovascular event affecting cognition and/or significant cardiovascular risk factors considered to be related to the cognitive decline, are included. Such mixed patients may also have pathology and/or symptoms associated with other dementias, such as AD, Huntington’s disease, autism spectrum disorder, Down syndrome, progressive supranuclear palsy, corticobasal degeneration, Parkinson’s Disease, amyotrophic lateral sclerosis, Dementia with Lewy Bodies, Frontotemporal Dementia, normal pressure hydrocephalus and head injuries, among others.

Imaging is as useful tool in diagnosing dementia, in particular computerized tomography (CT), magnetic resonance imaging (MRI) and positron emission spectroscopy (PET). Neural degeneration results in brain atrophy and this can be detected and quantified. Patients treatable according to the invention may show global brain atrophy, measurable on the global cortical atrophy (GCA) scale. A score of 1 on the scale may be considered normal in an elderly patient, but scores of 2 or 3 should generally be considered to be abnormal. Subjects with a GCA score of 2 or 3 are preferably treatable according to the invention. Severe cases of atrophy may show pronounced ventricular enlargement and such patients are suitably treated using the inventive methods. Asymmetric and/or regional atrophy detected by MRI, particularly of the temporal and/or parietal regions, is highly suggestive of AD. Automated tools are increasingly available that can perform these functions in order to detect abnormal brain atrophy indicative of AD.

Fluorodeoxyglucose (FDG) PET scans measure glucose use in the brain. Glucose, a type of sugar, is the primary source of energy for cells. Studies show that people with dementia often have abnormal patterns of decreased glucose use in specific areas of the brain. An FDG PET scan can show a pattern that may support a diagnosis of a specific cause of dementia. The invention contemplates treating patients with evidence of AD pathology detected by PET, including but not limited to FDG PET. FDG PET detects regions of glucose hypometabolism, indicating metabolic impairment.

Amyloid PET scans measure abnormal deposits of a protein called beta-amyloid and can be used to identify patients having AD pathology, whether pure AD, which is excluded from the invention, or mixed VaD/AD, which is included. Higher levels of beta-amyloid are consistent with the presence of amyloid plaques, a hallmark of Alzheimer’s disease. Several tracers may be used for amyloid PET scans, including florbetapir, flutemetamol, florbetaben, and Pittsburgh compound B. The invention contemplates treating mixed dementia patients with evidence of amyloid deposits by PET scan using on or more of the foregoing tracers.

Tau PET scans detect abnormal accumulation of a protein, tau and can be used to identify patients having AD pathology, whether pure AD, which is excluded from the invention, or mixed VaD/AD, which is included. Tua forms tangles in nerve cells in Alzheimer’s disease and many other dementias, like frontotemporal dementia. Several tau tracers, such as AV-1451, PI-2620, and MK-6240, are being studied in clinical trials and other research settings. The invention contemplates treating mixed dementia patients with evidence of NFTs by PET scan using on or more of the foregoing tracers.

Regional hypoperfusion is also associated with functional deficits seen in dementia. Hypoperfusion may be detected by a number of methodologies, including spin-labeling MRI and single-photon emission computed tomography (SPECT). The invention contemplates treating patients with evidence of regional hypoperfusion, detected by spin-labeling MRI, SPECT and other methods known to the skilled artisan.

Patients treatable according to the invention will typically score poorly on cognitive scales, such as the mini mental state exam (MMSE). A threshold of ≤ 23 on the MMSE is set for dementia, with score of ≤15 Representing severe dementia. Patients with an MMSE score of 24 - 27 are considered to have mild cognitive impairment. Patients may have mild cognitive impairment (MMSE 24-27), but patients treated according to the invention preferably have an MMSE score of less than 23 and some patients have a minimum MMSE of 15. In certain aspects of the invention treated patients will have an MMSE score of ≤ 20 or ≤18 or ≤ 16. Once the MMSE falls below 15, the Severe Impairment Battery (SIB) is a useful assessment too.

Other short tools for assessing dementia/diminished cognition and for measuring cognitive improvement include: the Eight-item Informant Interview to Differentiate Aging and Dementia (AD8); the Annual Wellness Visit (AWV); the General Practitioner Assessment of Cognition (GPCOG); Health Risk Assessment (HRA); Memory Impairment Screen (MIS); the Montreal Cognitive Assessment (MoCA); the St. Louis University Mental Status Exam (SLUMS); and the Short Informant Questionnaire on Cognitive Decline in the Elderly (Short IQCODE).

Another useful scale for measuring some of the symptoms of dementia is the Cohen-Mansfield Agitation Inventory (CMAI).

The CDR Dementia Staging Instrument is a 5-point scale used to characterize six domains of cognitive and functional performance in AD: Memory, Orientation, Judgment & Problem Solving, Community Affairs, Home & Hobbies, and Personal Care. It is scored according to the following scale: 0 = Normal; 0.5 = Very Mild Dementia; 1 = Mild Dementia; 2 = Moderate Dementia; 3 = Severe Dementia. Patients treatable according to the invention will preferably have a CDR score of 2 or 3. The CDR is generally scored according to an algorithm the differentially weighs the sub-scores (0, 0.5, 1, 2 or 3) from the various domains. The CDR may also be scored in an alternative manner which simply adds up the sub-scores for each domain. The so-called sum-of-boxes (SOB) method is equally valid, but has higher resolution, yielding scores from 0 (normal) to 18 (score of 3 on every domain). Patients treatable according to the invention will generally score a minimum of 4.5 using the CDR-SOB scoring.

Some aspects of the invention improve wandering symptoms. Wandering generally can be characterized by two domains. The first domain is movement, generally in the form of ambulation unless the patient is disabled and, for example, confined to a wheelchair. The second domain is problematic behavior, usually in the form of boundary transgressions and/or wayfinding problems. However, it could be reflected in the movement itself, such as pacing or lapping behavior. It may involve inappropriately following a caregiver. A common problematic behavior is attempted escape or elopement. A certain quantity of movement may also be considered the problematic behavior. A normal person is in motion approximately 10% of their waking hours and so movement beyond this threshold amount can be considered problematic behavior. A patient will be considered to suffer from wandering when in motion for at least 20% of their waking hours, but preferably more than 30% of their waking hours. As a patient spends more time in motion, the behavior becomes particularly problematic because they risk exhaustion and, therefore, falling and serious injury. Thus, some wandering patients are in motion more that 40% or 50% of their waking hours and some more than 60%, 70% or even 80%.

It has been proposed that wandering can be persistent or sporadic and the present methods may be used to treat either population. Persistent wanderers exhibit excessive movement nearly every day, typically at least 4 - 5 days per week. On the other hand, sporadic wanderers do not exhibit excessive movement, but rather they are generally sedentary with occasional movement, typically associated with elopement, boundary transgressions, escape or wayfinding defects. Sporadic wanders may exhibit the behavior as infrequently as monthly or as frequently as 2, 3 or even 4, 5, 6, or more times per week. Unlike the persistent wanderer, the sporadic wonderer does not spend an abnormally high amount of time in motion. In one preferred embodiment of the invention, patients treated wander due to dementia of any form and do not display a wayfinding defect; such a patient may be a persistent or a sporadic wanderer.

### Dosing Regimens

In accordance with the treatment methods of the present invention, administering a therapeutically effective amount of a ROCK inhibitor or a pharmaceutically acceptable salt thereof one or more times a day. The lowest therapeutically effective amount of fasudil, for example, is 70 mg per day, generally administered in 2 to 3 equal portions to obtain the full daily dose. The highest therapeutically effective dose may be determined empirically as the highest dose that remains effective in alleviating one or more dementia-related signs or symptoms, but does not induce an unacceptable level or adverse events. Fasudil, for example, generally will not be administered in a daily dose exceeding 180 mg. One preferred dosing regimen involves the treatment with 25, 30, 40 or 60 mg of Fasudil hydrochloride hemihydrate three times per day using an immediate-release formulation, for a total daily dose of 75 - 180 mg. Preferred dosing exceeds a daily dose of 70 mg, with most preferred ranges for daily dosing being 70 mg to 140 mg administered in three equal amounts during the day. Other preferred daily doses will range from 90 mg to 180 mg per day or 80 mg to 150 mg per day. A further dosing regimen involves the treatment with, 35 to 90 mg of Fasudil hydrochloride hemihydrate only two times per day using an immediate-release formulation, for a total daily dose of 70 - 180 mg. Generally, an oral daily dose of 70 - 75 mg will the minimum required to see a treatment effect. At more than 180 mg per day given orally, kidney function begins to be affected and higher dosing in most patients will not be warranted. Above 240 mg per day, kidney effects of the drug are generally unacceptable. Based on ROCK inhibitory activity, one skilled in the art can readily extrapolate the provided dosing ranges for fasudil to other ROCK inhibitors.

The treatment methods of the present invention, while contemplating various routes of administration, are particularly suited to oral administration. Thus, it will be understood that an effective amount of a ROCK inhibitor or a pharmaceutically acceptable salt thereof preferably is administered orally one or more times orally per day and an effective amount may range from the lowest therapeutically effective amount of fasudil, which is 70 mg per day. Generally, it will be administered orally in 2 to 3 equal portions to obtain the full daily dose. The daily oral dose of fasudil, for example, generally will not exceed 180 mg. One preferred dosing regimen involves the treatment with 25, 30, 40 or 60 mg of Fasudil hydrochloride hemihydrate three times per day orally using an immediate-release formulation, for a total daily dose of 75 - 180 mg. Preferred dosing exceeds a oral daily dose of 70 mg, with most preferred ranges for daily dosing being 70 mg to 140 mg administered in three equal amounts orally during the day. Other preferred daily doses will range from 90 mg to 180 mg per day or 80 mg to 150 mg orally per day. A further dosing regimen involves the treatment with, 35 to 90 mg of Fasudil hydrochloride hemihydrate only two times per day using an immediate-release oral formulation, for a total daily dose of 70 - 180 mg. Generally, an oral daily dose of 70 - 75 mg will the minimum required to see a treatment effect. At more than 180 mg per day given orally, kidney function begins to be affected and higher dosing in most patients will not be warranted. Above 240 mg per day orally, kidney effects of the drug are generally unacceptable. Based on ROCK inhibitory activity, one skilled in the art can readily extrapolate the provided dosing ranges for fasudil to other ROCK inhibitors.

Certain patient sub-populations, such as renally impaired patients and/or older patients (e.g., 65 or older) may need lower doses or extended release formulations instead of immediate release formulations. Fasudil hydrochloride hemihydrate may have higher steady-state concentrations when given at usual doses to patients with renal disease and lower doses to lower the Cmax or delay the time to Cmax (increase the Tmax) may be required.

Renal dysfunction occurs with age and as the result of numerous disorders, including liver cirrhosis, chronic kidney disease, acute kidney injury (for example, due to administering a contrast agent), diabetes (Type 1 or Type 2), autoimmune diseases (such as lupus and IgA nephropathy), genetic diseases (such as polycystic kidney disease), nephrotic syndrome, urinary tract problems (from conditions such as enlarged prostate, kidney stones and some cancers), heart attack, illegal drug use and drug abuse, ischemic kidney conditions, urinary tract problems, high blood pressure, glomerulonephritis, interstitial nephritis, vesicoureteral, pyelonephritis, sepsis. Kidney dysfunction may occur in other diseases and syndromes, including non-kidney-related diseases that may occur along with kidney dysfunction, for example pulmonary artery hypertension, heart failure, and cardiomyopathies, among others.

Kidney function is most often assessed using serum (and/or urine) creatinine. Creatinine is a breakdown product of creatine phosphate in muscle cells and it is produced at a constant rate. It is excreted by the kidneys unchanged, principally through glomerular filtration. Accordingly, elevated serum creatinine is a marker for kidney dysfunction and it is used to estimate glomerular filtration rate.

Normal levels of creatinine in the blood are approximately 0.6 to 1.2 mg/dL in adult males and 0.5 to 1.1 mg/dL in adult females. When creatinine levels exceed these figures, the subject has renal dysfunction, and is, therefore, treatable according to the invention. Mild renal impairment/dysfunction occurs in the range of 1.2 mg/dL to 1.5 mg/dL. Moderate renal impairment/dysfunction is considered to occur at creatinine levels exceeding 1.5 mg/dL. Severe renal impairment, which includes what is considered to be renal failure, is defined as a serum creatinine level of ≥ 2.0 mg/dL or the use of renal replacement therapy (such as dialysis). Treating subjects with mild, moderate and severe renal impairment is specifically contemplated.

As indicated, creatinine levels are considered to be a surrogate for glomerular filtration rate (GFR) and serum creatinine levels alone may be used to estimate glomerular filtration rate using the Cockroft-Gault equation.

According to the National Kidney Foundation, the following GFRs indicate the varying levels of renal function:

| GFR (ml/min/1.73 m²) | Renal Function |
|---|---|
| ≥90 | Normal or high |
| 60-89 | Mildly decreased |
| 45-59 | Mildly to moderately decreased |
| 30-44 | Moderately to severely decreased |
| 15-29 | Severely decreased |
| <15 | Kidney failure |

In general, creatinine clearance (estimated glomerular filtration rate) may be derived directly from serum creatinine using the Cockroft - Gault equation:

$$\begin{matrix}\text{creatinine clearance =} \left(\left(\left(\text{140 - age in years}\right) \times \left(\text{wt in kg}\right)\right) \times \text{1} \text{.23}\right) / \\ \left(\text{serum creatinine in} μ \text{} \text{mol} / \text{L}\right)\end{matrix}$$

For women the result of the calculation is multiplied by 0.85.

Empirically measured creatinine clearance may also be used directly as an estimate of glomerular filtration rate by looking at serum creatinine and urine creatinine levels. Specifically, urine is collected over 24 hours and the following equation is applied to ascertain creatinine clearance:

$$\begin{matrix}\text{Creatinine Clearance} \left(\text{mL} / \text{min}\right) = \text{Urine Creatinine Concentration} \\ \left(\text{mg} / \text{mL}\right) * 24 \text{hour urine volume} \left(\text{mL}\right) / \text{Plasma Creatinine} \\ \text{Concentration} \left(\text{mg} / \text{mL}\right) * 24 \text{hour} * 60 \text{minutes}\end{matrix}$$

In one embodiment, dose of fasudil for mild to moderate renal impairment is reduced to 50-80 mg per day. In another embodiment, the dose of fasudil is not reduced but is administered one time per day in an extended release dosage form.

In another embodiment, the dose is not reduced for mild to moderate renal impairment.

In one embodiment, the dose of fasudil is reduced to 30-45 for severe renal impairment. In another embodiment, the dose of fasudil is not reduced but is instead administered one time per day in an extended release dosage form.

In a further embodiment, the dose is reduced where serum creatinine (SCr) >2 and/or an increase in SCr > 1.5x from baseline, and/or a decrease in eGFR >25% from baseline.

Patient size is an important factor to consider when using creatinine-based estimates of renal function. The units of drug clearance are volume/time (mL/min), whereas the units of estimated GFR for chronic renal disease are volume/time/standard size (mL/min/1.73 m²). Generally, doses may be adjusted down (e.g., 40-50 mg per day) for smaller patients and up for larger (e.g., 120 mg per day) for obese patients. A smaller male would be about 160 pounds or less. A smaller female patient would weigh about 130 pounds or less. Patients having a Body Mass Index of 30 and higher is considered obese.

In addition, older patients may need a lower dose at initiation, with a gradual increase to the recommended dose after days or weeks. In another embodiment, older patients may need lower doses for the duration of treatment. The aged population includes the “young old” who are 65-74, the “old old” who are 75-84 and the “frail elderly” who are 85 and older. For example, a starting dose of 30 mg per day for two weeks, followed by 60 mg per day for 4 weeks, then by 90 mg per day. Titration may even be warranted up to about 120 mg per day.

Another embodiment involves the treatment with 60-120 mg of fasudil hydrochloride hemihydrate once per day in an extended release dosage form. Treatment with an extended release total daily dose of 90 mg fasudil hydrochloride hemihydrate once per day is preferred. It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

Methods of administering compositions according to the invention would generally be continued for at least one day. Some preferred methods treat for up to 30 days or up to 60 days or even up to 90 days or even more. Treatment for more than 60 days is preferred and treatment for at least 6 months is particularly preferred. The precise duration of treatment will depend on the patient’s condition and response to treatment. Most preferred methods contemplate that treatment begins after the onset or appearance of symptoms.

The methods of the invention also contemplate administering ROCK inhibitors with other compounds used to treat dementia or other symptoms of dementia. They may be administered in combination, a single dosage form, in a common dosing regimen or administered to the same patient at different times of the day using different dosing regiments.

Two classes of drugs are used to treat dementia and have been shown to improve cognition: acetylcholinesterase inhibitors and N-methyl-D-aspartate (NMDA) receptor antagonists. Generally used in the early stages of disease, acetylcholinesterase inhibitors prevent the breakdown of the neurotransmitter acetylcholine. These drugs include piperidines like donepezil (Aricept), phenanthrene derivatives, like galantamine (Razadyne), and carbamates like rivastigmine (Exelon). NMDA receptor antagonists include the uncompetetitive inhibitor memantine (Namenda). A combination of memantine and donepezil (Namzaric) is also available.

In some embodiments, the patients are administered fasudil in combination with other actives approved to treat dementia, including but not limited to cholinesterase inhibitors and NMDA receptor antagonists. In one embodiment, the cholinesterase inhibitor is selected from the group consisting of donepezil, rivastigmine, and galantamine. Exemplary doses of the cholinesterase inhibitors include 3-25 mg per day, more preferably 6-12 mg per day. In another embodiment, the NMDA receptor antagonist is memantine. In a specific embodiment, memantine is administered at a dose of 5-28 mg per day, preferably 15-20 mg per day. In a further embodiment, the co-administered active is a combination of donepezil and memantine at a dose of 28 mg memantine and 10 mg donepezil.

In a specific embodiment, the combination of fasudil with cholinesterase inhibitors is administered to AD patients. In a further embodiment, the combination of fasudil with cholinesterase inhibitors is administered to patients with mixed dementia that is predominantly of the AD type. In yet a further embodiment, the combination of fasudil with cholinesterase inhibitors is not administered to patients only vascular dementia.

Dextromethorphan hydrobromide is another an uncompetitive NMDA receptor antagonist that also has activity as a sigma-1 receptor agonist. Marketed in combination quinidine sulfate (a CYP450 2D6 inhibitor), the product Nudexta is indicated for the treatment of pseudobulbar affect, which occurs in many forms of dementia. In one embodiment, a patient is treated with product useful in treating pseudobulbar affect, like Nudexta, and fasudil.

In a further embodiment, the patient treated with fasudil is also being treated with active agents including mood stabilizers, benzodiazepines, antipsychotics, anti-agitation drugs, or sleep aids. In a specific embodiment, the patient treated with fasudil is not being treated with risperidone, aripiprazole, quetiapine, carbamazepine, gabapentin, prazocin, trazodone or lorazepam.

In a further embodiment the patient treated with fasudil is being treated for depression. In a specific embodiment, the patient is treated with an anti-depressant such as citalopram or escitalopram.

Dextromethorphan hydrobromide is another an uncompetitive NMDA receptor antagonist that also has activity as a sigma-1 receptor agonist. Marketed in combination quinidine sulfate (a CYP450 2D6 inhibitor that prolongs the half-life of dextromethorphan), the product Nudexta is indicated for the treatment of pseudobulbar affect, which occurs in many forms of dementia.

The methods of the invention in certain embodiments, especially those contemplating parenteral dosing, do not include the administration of a statin (rosuvastatin, especially) to a patient also receiving a rho kinase inhibitor. The methods of the invention in certain embodiments, especially those contemplating parenteral dosing, do not include the administration of nimodipine to a patient also receiving a rho kinase inhibitor.

### Results of the Methods

The methods of the invention are considered to be disease modifying, such that they will result in improvements in all related signs and symptoms. Such improvements may be absolute, in that a treated patient will actually show an improvement over time relative to a previous measurement. Improvements are more typically measured relative to control patients. Control patients may be historical and/or based on the known natural history of similarly-situated patients, or they may be controls in the sense that they receive placebo or simply standard of care in these same clinical trial. Comparison to controls is especially instructive as it is unlikely that the course of the disease will be fully reversed and so results are measure in terms of decreased deterioration relative to controls/expectations.

Improvements can be assessed using one or more of the following scales: the MMSE; the SIB; the AD8; the AWV; the GPCOG; the HRA; the MIS; the MoCA; the SLUMS; the Short IQCODE; the CDR; the ADAS-Cog; the ADCS-CGIC; and the CMAI, including variants thereof.

Improvements resulting from the inventive methods will generally be at least 10%; 15%; 20%; 25%; 30%; 35%; 40%; 45% or 50%, absolute or in comparison to a control. In another embodiment, improvements resulting from the inventive methods will be at least 50% or more, absolute or in comparison to a control. In preferred embodiments, improvements resulting from the inventive methods will be at least 75%, absolute or relative to a control.

Treatment using the inventive methods generally result in improved cognitive functioning. Patients will generally show improvement on the MMSE and/or the SIB of at least 3 points during the early stages of treatment and declines in cognition are slowed relative to control patients, generally maintaining at least a 1- or 2-point differential in treated and control patients.

A typical patient treated according to the invention may show improvements of at least 0.5 points on the CDR-SOB, but in any event will show a reduced rate of decline, manifesting as at least a 1-point differential on the CDR-SOB versus untreated controls after treatment for at least 6 months.

Patient treated according to the invention are also expected to show improvements in one or more of the following: forgetfulness, slowing of thought processes, mild intellectual impairment, apathy, inertia, depression, irritability, loss of recall ability, and the inability to manipulate knowledge, mood disorders, repetitive behavior, compulsive behavior, defects in executive function, deficits in speed, deficits in attention, deficits in planning, deficits in monitoring, deficits in memory tasks, aphasia, apraxia, amnesia, recall abnormality, deficits in encoding information, deficits in memory consolidation, lack of spontaneity, perseveration, and/or deficits in spontaneous recall.

In one specific embodiment, treatment with fasudil reduces the amount of repetitive movement wandering (e.g., lapping, pacing) in the patients by at least 10%; 15%; 20%; 25%; 30%; 35%; 40%; 45% or 50%. In another embodiment, treatment with fasudil reduces the amount repetitive movement wandering by 50% or more. In preferred embodiments, treatment with fasudil reduces repetitive movement by at least 75%. In a preferred embodiment, treatment with fasudil reduces the amount of repetitive movement wandering to the normative 10% motion during waking hours.

In a further embodiment, treatment with fasudil reduces the number of times per day repetitive movement wandering occurs by at least one time, preferably by at least two times, and more preferably by at least three times per day.

In a further embodiment, treatment with fasudil reduces the number of days repetitive movement wandering occurs by at least one day per week, preferably by at least two days per week, and more preferably by at least three days per week.

In another specific embodiments, treatment with fasudil reduces persistent wandering by at least 10%; 15%; 20%; 25%; 30%; 35%; 40%; 45% or 50%. In another embodiment, treatment with fasudil reduces persistent wandering by 50% or more. In preferred embodiments, treatment with fasudil hydrochloride hemihydrate reduces persistent wandering by at least 75%. In a preferred embodiment, treatment with fasudil reduces persistent wandering to the normative 10% motion during waking hours.

In a further embodiment, treatment with fasudil reduces the number of days wandering occurs in persistent wandering by at least one day per week, preferably by at least two days per week, and more preferably by at least three days per week.

In another embodiment, treatment with fasudil reduces sporadic wandering by at least 10%; 15%; 20%; 25%; 30%; 35%; 40%; 45% or 50%. In another embodiment, treatment with fasudil reduces sporadic wandering by 50% or more. In preferred embodiments, treatment with fasudil reduces sporadic wandering by at least 75%. In a preferred embodiment, treatment with fasudil reduces sporadic wandering to the normative 10% motion during waking hours.

In another embodiment, treatment with fasudil reduces pacing or lapping by at least 10%; 15%; 20%; 25%; 30%; 35%; 40%; 45% or 50%. In another embodiment, treatment with fasudil reduces pacing or lapping by 50% or more. In preferred embodiments, treatment with fasudil reduces pacing or lapping by at least 75%.

In another embodiment, treatment with fasudil reduces eloping behavior by at least 10%; 15%; 20%; 25%; 30%; 35%; 40%; 45% or 50%. In another embodiment, treatment with fasudil reduces eloping behavior by 50% or more. In preferred embodiments, treatment with fasudil reduces eloping behavior by at least 75%.

In another embodiment, treatment with fasudil reduces spatial disorientation by at least 10%; 15%; 20%; 25%; 30%; 35%; 40%; 45% or 50%. In another embodiment, treatment with fasudil reduces spatial disorientation by 50% or more. In preferred embodiments, treatment with fasudil reduces spatial disorientation by at least 75%.

In another embodiment, treatment with fasudil reduces the caregiver burden associated with wandering by at least 10%; 15%; 20%; 25%; 30%; 35%; 40%; 45% or 50%. In another embodiment, treatment with fasudil reduces the caregiver burden associated with wandering by 50% or more. In preferred embodiments, treatment with fasudil reduces the caregiver burden associated with wandering by at least 75%.

In another embodiment, treatment with fasudil reduces the caregiver burden associated with one or more of persistent wandering, pacing, elopement and spatial disorientation by at least 10%; 15%;20%; 25%; 30%; 35%; 40%; 45% or 50%. In another embodiment, treatment with fasudil reduces the caregiver burden associated with one or more of persistent wandering, pacing, elopement and spatial disorientation by 50% or more. In preferred embodiments, treatment with fasudil reduces the caregiver burden associated with one or more of persistent wandering, pacing, elopement and spatial disorientation by at least 75%.

In a further embodiment, treatment with fasudil reduces the number of days wandering occurs in sporadic wandering by at least one day per week, preferably by at least two days per week, and more preferably by at least three days per week.

In another embodiment, treatment with fasudil reduces the wandering occurs during sundowning, or early evening. In another embodiment, treatment with fasudil reduces the wandering occurs during the overnight hours. In one embodiment, the amount of wandering to determine the reduction can be measured using electronic motion and/or activity tracking device, including fitness trackers such as Fitbits. The fitness trackers can be used alone or in combination with GPS devices to measure location.

The Revised Algase Wandering Scale (Long Term Care Version) is a preferred instrument for measuring wandering (Nelson and Algase 2006). It is divided into three different domains based on the three main wandering typologies: Persistent Wandering (PW); Eloping Behavior (EB); and Spatial Disorientation (SD). Each domain evaluates individual items on a scale that can be quantified with a score from 1-4.

An overall domain score is calculated based on the number of questions with a valid response. Thus, the individual scores are added up and divided by the number of questions in the domain with valid responses. It is highly preferred that at least 75% of the items in a domain have valid responses. The result will be a score from 1 to 4.

Likewise, an overall scale score may be obtained by averaging each of the 3 domains, resulting in a global score of 1-4. Alternatively, for the highest level of granularity, each individual item within a domain may be assessed individually.

The RAWS can be filled out by staff or a caregiver.

The PW domain consists of 9 individual items that look at the amount of spontaneous walking in absolute terms and relative to other similarly situated patients, pacing and restless walking (which may indicate agitation) and the timing of the wandering relative to mealtimes, which may be indicative of provocation to wander.

The EB domain consists of 4 items. It measures running off, entering unauthorized areas, leaving authorized areas and returns to authorized areas after an unnoticed leaving.

The SD domain consists of 6 items that assess getting lost, aimless walking, running into people and objects and the inability to locate certain rooms.

In certain embodiments, patients treated according to the invention will show improvements in at least 1 item of the RAWS. In preferred embodiments, patients will show improvements in at least one domain of the RAWS. In particularly preferred embodiments, patients will show improvements in the PW and/or the EB domain of the RAWS. Such improvements will generally be in the range of 10%; 15%; 20%; 25%; 30%; 35%; 40%; 45% or 50%.

### EXAMPLE

Eighty patients diagnosed with VaD or mixed dementia with VaD are recruited. Patients with no apparent vascular etiology or pathology are excluded. Patients with a non-neurological comorbidity or who use medication that could adversely affect cognition are also excluded. Patients have a maximum MMSE score of 23 and a minimum MMSE score of 15.

Cohorts of 20 patients are treated orally with fasudil or placebo in a dose escalating manner. Each group is randomized 10 patients each to placebo or drug and treated for 60 days. At the end of 30 days, based on assessment of adverse event, the next cohort with a higher dose is begun. At the end of 60 days, patients will be assessed for efficacy and safety and will be re-randomized into the next higher dose in the absence of dose-limiting side effects. Oral dosing using 10 mg immediate release tablets starts with the first cohort at 60 mg per day(administered in 3 equal doses throughout the day), the second cohort at 90 mg per day (administered in 3 equal doses throughout the day), the third cohort at 180 mg per day (administered in 3 equal doses throughout the day) and the fourth cohort at the maximum planned dose is 240 mg per day (administered in 3 equal doses throughout the day).

No effect in cognition is observed with the 60 mg dose at 60 days, whereas each of the other doses show improvements at 60 days versus control. When the first cohort is escalated to 90 mg per day, a difference in cognition between treated and control in that cohort is observed. Cognition improves in a dose-dependent manner across all doses. A dose-dependent increase in creatinine, indicating possible kidney dysfunction is seen. Only 50% of the subjects who are escalated to the 180 mg per day dose are also escalated to the 240 mg dose and 25% of patients treated with 240 mg daily are dose-reduced due to elevated creatinine levels.

It is determined that the optimal dose for improving cognition in AD dementia is between 90 mg and 120 mg per day. Below 90 mg, there is no efficacy and above 120 mg elevated creatinine becomes dose-limiting in many patients.

### LIST OF REFERENCES

Becker RE, Greig, NH, Giacobini E, Why Do So Many Drugs for Alzheimer’s Disease Fail in Development? Time for New Methods and New Practices?, Alzheimers Dis. 15:303-325 (2008)

Ceyzériat et al., Current Alzheimer Research 17: 1-13 (2020)

Chen M, Liu A, Ouyang Y, Huang Y, Chao X, Pi R. 2013. Fasudil and its analogs: a new powerful weapon in the long war against central nervous system disorders? Expert Opin Investig Drugs. 22:537-50.

Couch BA, DeMarco GJ, Gourley SL, Koleske AJ, Increased Dendrite Branching in AβPP/PS1 Mice and Elongation of Dendrite Arbors by Fasudil Administration. Alzheimers Dis. 2010 ; 20(4): 1003-1008.

Elliott C, Rojo A, Ribe E, Broastock M, Xia W, Morin P, Semenov M, Baillie G, Cuadrado A, Al-Shawi R, Ballard C, Simons P, Killick R, A role for APP in Wnt signalling links synapse loss with β-amyloid production. Translational Psychiatry. 2018; 8(179).

Erkinjuntti T, Inzitari D, Pantoni L, Wallin A, Scheltens P, Rockwood K, Desmond DW. 2000. Limitations of clinical criteria for the diagnosis of vascular dementia in clinical trials. Is a focus on subcortical vascular dementia a solution? Ann N Y Acad Sci. 903:262-72.

Feng Y, LoGrasso P, Defert O, Li R, Rho Kinase (ROCK) Inhibitors and Their Therapeutic Potential. J Med Chem. 2016; 59*6): 2269-2300.

Folstein MF, Folstein SE, McHugh PR. “Mini-mental state”: a practical method for grading the cognitive state of patients for the clinician. J Psychiatr Res. 1975;12:189-198.

Hamano T, Shirafuji N; Yen S; Yoshida H, Kanaan N, Hayashi K, Ikawa M, Yamamura O, Fujita Y; Kuriyama M, Nakamoto Y, Rho-kinase ROCK inhibitors reduce oligomeric tau protein. Neurobiology of Aging; 2020; 89: 41-54.

Hou Y, Zhou L, Yang QD, Du XP, Li M, Yuan M, Zhou ZW, Changes in hippocampal synapses and learning-memory abilities in a streptozotocin-treated rat model and intervention by using fasudil hydrochloride. Neuroscience. 2012; 200: 120-129.

Jacobs M, Hayakawa K, Swenson L, Bellon S, Fleming M, Taslimi P, Doran J, The structure of dimeric ROCK I reveals the mechanism for ligand selectivity. J Biol Chem. 2006; 281(1): 260-68.

Kamei S, Oishi M, Takasu T. 1996a. Evaluation of fasudil hydrochloride treatment for wandering symptoms in cerebrovascular dementia with 31P-magnetic resonance spectroscopy and Xe-computed tomography. Clin Neuropharmacol. 19:428-38.

Kamei S, Toshiaki T, Oishi M, Effect of fasudil hydrochloride on wandering symptoms of c cerebrovascular dementia patients. Neurotherapy. 1996b 13:43-50.

Kim KW, Lee DY, Jhoo JH, Youn JC, Suh YJ, Jun YH, Seo EH, Woo JI, Diagnost accuracy of mini-mental status examination and revised Hasegawa dementia scale for Alzheimer’s disease. Dement Geriatr Cogn Disord. 2005;19(5-6):324-30.

Nakagawa O, Fukisawa K, Ishizaki T, Saito Y, Nakao K, Narumiya S, ROCK-I and ROCK-II, two isoforms of Rho-associated coiled-coil forming protein serine/threonine kinase in mice. FEBS Lett. 1996 Aug 26;392(2):189-93.

Nair & Jacob, A simple practice guide for dose conversion between animals and human. J Basic Clin Pharm. 7:27-31 (2016).

Nelson & Algase (Eds) Evidence-based Protocols for Wandering Behaviour (2006), Springer:NY. Risk Model.

Román GC, Tatemichi TK, Erkinjuntti T, Cummings JL, Masdeu JC, Garcia JH, Amaducci L, Orgogozo JM, Brun A, Hofman A, et al. 1993. Vascular dementia: diagnostic criteria for research studies. Report of the NINDS-AIREN International Workshop. Neurology. 43:250-60.

Román GC. 2004. Facts, myths, and controversies in vascular dementia. J Neurol Sci. 226:49-52.

Salardini A. An Overview of Primary Dementias as Clinicopathological Entities. Semin Neurol. 2019; 39:153-166.

Sasaguri H, Nilsson P, Hashimoto S, Nagata K, Saito T, De Strooper B, Hardy J, Vassar R, Winblad B, Saido TC, APP mouse models for Alzheimer’s disease preclinical studies. EMBO J. 2017; 36(17): 2473-2487.

Sellers K, Elliott C, Jackson J, Ghosh A, Ribe E, Rojo A, Jarosz-Griffiths HH, Watson AA, Xia W, Semenov M, Morin P, Hooper N, Porter R, Preston J, Al-Shawi R, Baillie G, Lovestone S Cuadrado A, Harate M, Simons P, Srivastava DP, Killick R, Amyloid ß synaptotoxicity is Wnt-PCP dependent and blocked by fasudil. Alzeimer’s & Dementia. 2018; 14: 306-317.

Shibuya M, Asano T, Sasaki Y. 2001. Effect of Fasudil HCl, a protein kinase inhibitor, on cerebral vasospasm. Acta Neurochir Suppl. 77:201-4.

Turk M. The Effect of Rho Kinase Inhibitors on Alzheimer’s Disease, Dissertation. Arizona State University. May 2017.

Uehata M, Ishizaki T, Satoh H, Ono T, Kawahara T, Morishita T, Tamakawa H, Yamagami K, inui J, Maekawa M, Narumiya S, Calcium sensitization of smooth muscle mediated by a Rho-associated protein kinase in hypertension. Nature. 1997 Oct 30;389(6654):990-4.

Wetterling, R. D. Kanitz, and K. J. Borgis, “The ICD-10 criteria for vascular dementia,” Dementia. 1994; 5(3-4): 185-188.

Wetterling T, Kanitz RD, Borgis KJ. 1996. Comparison of different diagnostic criteria for vascular dementia (ADDTC, DSM-IV, ICD-10, NINDS-AIREN). Stroke. 27:30-6.

Yamaguchi H, Miwa Y, Kasa M, Kitano K, Amano M, Kaibuchi K, Hakoshima T, Structural basis for induced-fit binding of Rho-kinase to the inhibitor Y-27632. J Biochem. 2006 Sep;140(3):305-11.

Yu J, Gu Q, Yan Y, Yu H, Guo M, Liu C, Song G, Chai Z, Wang Q, Zia B, Zhang H, Jiang Y, Cungen MA, Fausidil improves cognition of APP/PS1 transgenic mice via inhibiting the activation of microglia and shifting microglia phenotypes from M1 to M2. Chin J Cell Mol Immunol. 2017; 33(12): 1585-1593.

Zhang X and Li L, Forty-five cases of therapeutic treatment for vascular dementia by fasudil hydrochloride. Herald of Medicine. 2012; 31(11): 1438-40.

The disclosure of each reference set forth herein is incorporated herein by reference in its entirety.

1. A method of treating vascular dementia, comprising administered to a patient suffering from vascular dementia a therapeutically effective amount of a rho kinase inhibitor.

2. The method according to claim 1, wherein the rho kinase inhibitor is fasudil and said therapeutically effective amount at least 70 mg daily, administered orally.

3. The method according to claim 1, wherein said method continues for a minimum of six months.

4. The method according to claim 1, wherein the upper dosing limit is determined by monitoring kidney function.

5. The method according to claim 1, wherein said therapeutically effective amount is no more than 120 mg per day, administered orally.

6. The method according to claim 1, wherein the patient has no evidence of a hemorrhagic lesion.

7. The method according to claim 1, wherein the patient does not have evidence of pseudobulbar affect.

8. The method according to claim 1, wherein the patient does not have hypertension.

9. The method according to claim 1, wherein cognition is improved following oral administration of the rho kinase inhibitor to a the patient .

10. The method according to claim 1, wherein executive is improved following oral administration of the rho kinase inhibitor to a the patient .

11. The method according to claim 1, wherein activities of daily living are improved following oral administration of the rho kinase inhibitor to a the patient suffering from vascular dementia a pharmacologically effective amount of a rho kinase inhibitor.

12. (canceled)

13. The method according to claim 1 wherein the rho kinase inhibitor is fasudil and fasudil is administered orally in a dose of between 70 and 140 mg per day in an immediate release formulation.

14. The method according to claim 1, wherein the patient has an MMSE score of.

15. The method according to claim 1, wherein the method continues for at least 6 months.

16. A method of reducing wandering in a patient with vascular dementia, comprising treating a patient with fasudil at an oral daily dose of at least 70 mg.

17. The method according to claim 16, wherein said wandering excludes wayfinding wandering.

18. The method according to claim 16 wherein that wandering is pacing or excess movement.

19. The method according to claim 16 wherein that wandering is elopement.

20. The method of treating cerebral autosomal dominant arteropathy with subcortical infarcts (CADASIL), comprising administered to a patient suffering from vascular dementia a therapeutically effective amount of a rho kinase inhibitor.

21. The method according to claim 20, wherein the rho kinase inhibitor is fasudil and said therapeutically effective amount at least 70 mg daily, administered orally.
